(19) United States
(12) Patent Application Publication
(10) Pub. No.: US 2010/0216251 A1
(43) Pub. Date: Aug. 26, 2010

(54) USE OF ACETIC NITRIC ACID REAGENT FOR EXTRACTION OF OLIGOSACCHARIDES AND POLYSACCHARIDES TO CHARACTERIZE CARBOHYDRATE MATERIALS FROM PLANTS AND OTHER SOURCES OF CELLULOSE USING GLYCAN OLIGOMER ANALYSIS

(76) Inventor: Allen Ketcik Murray, Newport Beach, CA (US)

Correspondence Address: ALLEN K. MURRAY, PH.D.;GLYCOZYME, INC., 17935 SKY PARK CIRCLE, STE. E, IRVINE, CA, 92614-6321, US

(21) Appl. No.: 12/448,746

(22) PCT Filed: Jan. 4, 2007
(86) PCT No.: PCT/IB2007/004690

§ 371 Date: Apr. 17, 2010

Related U.S. Application Data
(60) Provisional application No. 60/756,144, filed on Jan. 4, 2006.

(51) Int. Cl. G01N 33/50

(52) U.S. Cl. 436/94

(57) ABSTRACT

This patent application is for the use of acetic nitric reagent (80% acetic acid, 1.8 N nitric acid) for the extraction of oligosaccharides and polysaccharides from carbohydrate containing materials The material is extracted with the acetic nitric reagent in a boiling water bath for various periods of time, usually 30 minutes The material is then centrifuged and the clear, yellowish, supernatant is then taken to dryness in a Speed Vac under reduced pressure. The dry residue is then taken up in water and centrifuged to remove particulates The resulting supernatant is then analyzed by high pH anion exchange chromatography with integrated amperonetric detection The resulting chromatogram matogram or the integrated areas under the peaks are then characteristic for that particular source of material.

The following application is a continuation in part of and claims priority under 35 U.S.C. §119(e) from Provisional Patent Application No. U.S. 60/756,144 filed on Jan. 4, 2006, now abandoned.

### FIELD OF THE INVENTION

This invention involves a method of extraction of cell wall constitutents and using them to identify the origins of various plant cell walls. In particular this application describes biochemical methods of assessing the identity and quality of cotton fibers and of “fingerprinting” wood samples, food grains, foods derived from plant materials and any other material derived from a plant source.

### DESCRIPTION OF RELATED ART

This inventor has shown earlier that carbohydrate-containing cell wall fractions can be easily extracted from the lyophilized tissue by cold aqueous extraction and dilute acid extraction with HCl; then, subject the extracts to high pH anion exchange chromatography (HPAEC).

The use of HPAEC with integrated amperometric detection makes possible the unambiguous identification of cell wall constituents. In HPAEC a salt gradient (such as a sodium acetate gradient) is applied to a column of ion exchange resins held at a high pH to sequentially elute various mono and polysaccharides. Essentially, the hydroxyl groups of the sugars act as extremely weak acids that become deprotonated at the high pH, binding to the ion exchange matrix until eluted by the salt gradient.

While there are a number of vendors of HPAEC materials, the current invention has employed products and systems produced by the Dionex Corporation of Sunnyvale, Calif. These products and systems are explained in full in the Dionex Technical Notes, particularly in Technical Notes 20 and 21, which are hereby incorporated into this application. The carbohydrate fractions isolated from plant cell walls were analyzed using Dionex CarboPac PA1 and PA-100 columns. Both of these columns contain polystyrene/divinylbenzene cross-linked latex microbeads (350 nm diameter) with quaternary amine functional groups. The columns were operated under the manufacturer's recommended pressure conditions (4000 psi maximum) in sodium hydroxide eluted with a sodium acetate elution gradient When necessary, sugar alcohols were analyzed using a CarboPac MA1 column that contains porous beads (8.5 μm diameter) of vinylbenzene chloride/divinylbenzene with alkyl quaternary ammonium functional groups

The polysaccharides analyzed in the present invention are appropriately referred to as “glycoconjugates” because they comprise a monosaccharide conjugated to one or more additional monosaccharides (i.e., to form an oligo or polysaccharide) or sugar alcohol and optionally to a protein or a lipid. To summarize, glycoconjugates may be polysaccharides, polysaccharides containing a protein moiety, polysaccharides containing a lipid moiety and/or any combination of these. In any case HPAEC characterizes the polysaccharide component of the glycoconjugate.

### SUMMARY OF THE INVENTION

Not only are oligosaccharides and oligomers (multimers), which are chromatographic peaks eluting after a retention time of about 10 minutes, found in extracts of fibers sampled directly from cotton bolls, but extracts of cotton textiles produce peaks having the same retention times, relative to know compounds, as do the extracts of fibers from plant material. Moreover, the same oligosaccharides and oligomers can be recovered from cotton textiles. Similar oligosaccharides and oligomers may also be extracted from any cellulos containing material examples of some in this application are lotus seed coats and cotyledons, bamboo, regenerated cellulose sponge, bamboo fibers, regenerated bamboo fibers. While many of the same oligosaccharides and oligomers are found in the woods and in cotton, no two species of wood have been found to be display identical chromatograms. Furthermore, no two cultivars of cotton have been found to display identical chromatograms. Thus each species of wood has a distinct signature. For both cotton and wood, a probable hypothesis is that fractions of oligosaccharides and oligomers have leached out of the cellulose with successive exposure to water, detergent and salts. Loss of the oligosaccharides and oligomers may indicate, and may in fact constitute, wear and loss of integrity of the fabric and wood fibers.

Various cellulosic products also display oligosaccharides and oligomers similar to those found in cotton and wood. Every cellulosic product tested to date has produced a unique chromatogrm. The differences among the cellulose sources are probably due to differences in biochemistry and patterns of growth, the differences among the processed products may illustrate differences in both cellulose source and in processing. These differences are observed in the comparison of native bamboo fibers and regenerated bamboo fibers which yield remarkably different chromatograms.

### BRIEF DESCRIPTION OF THE DRAWINGS

FIG. 1. Acetic Nitric Extracts of Five Cultivars of cotton.

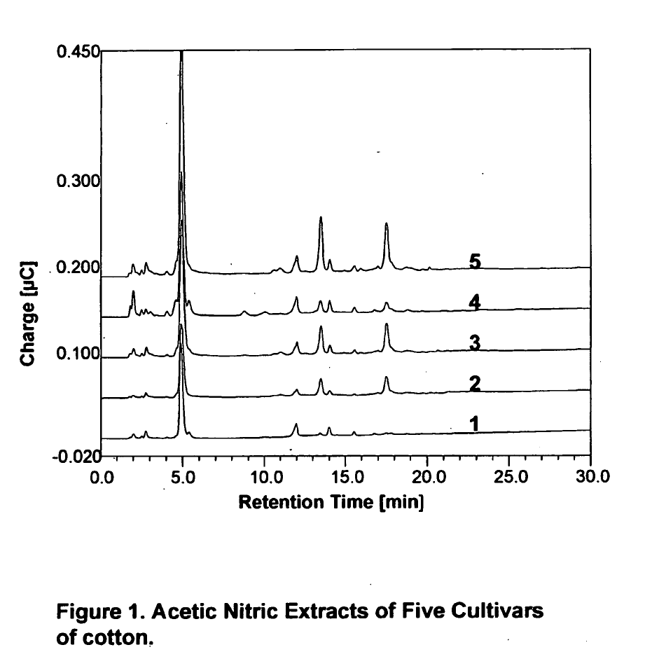

FIG. 2. Acetic Nitric Extracts of cotton linters, White Pine fibers and Avicel PH-101.

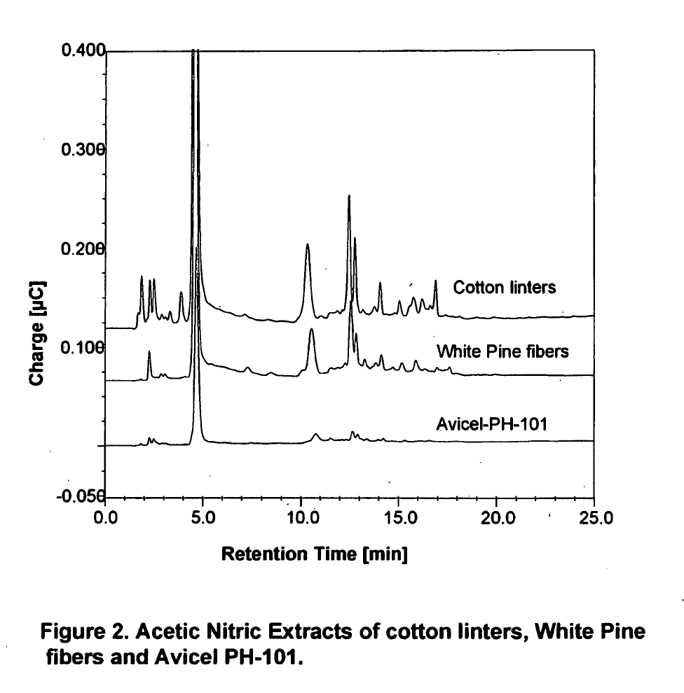

FIG. 3. Acetic Nitric Extracts of a knit cotton shirt and a cellulose (regenerated) sponge.

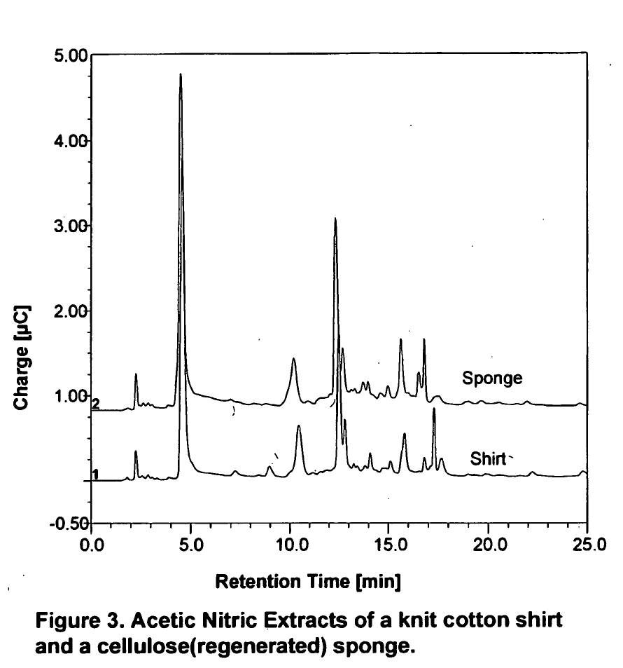

FIG. 4. Acetic Nitric Extracts of Bamboo fiber (natural), Coconut fiber, Ivory nut and Bamboo fiber (regenerated).

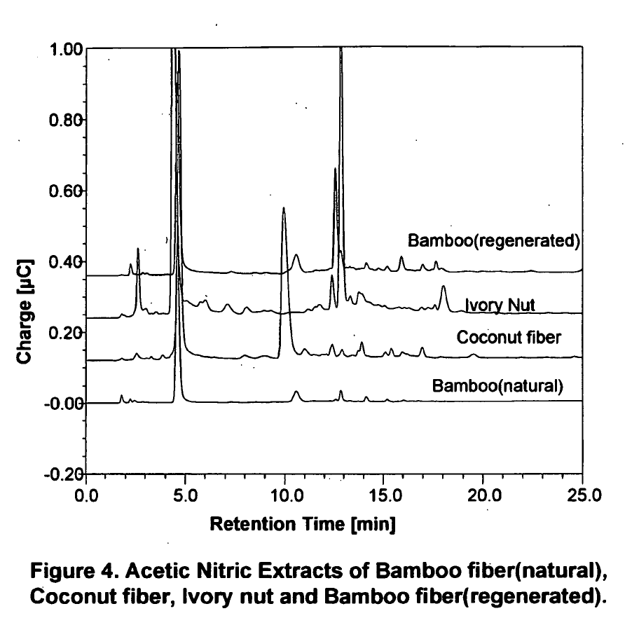

FIG. 5. Acetic Nitric Extracts of exudate gums of Yellow plum, Mariposa plum and Italian plum.

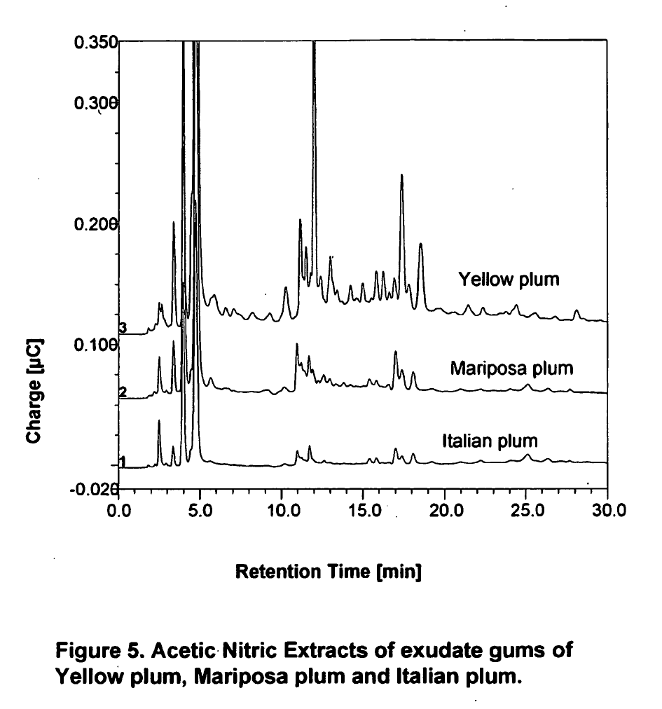

FIG. 6. Acetic Nitric Extracts of exudate gums of Bing Cherry, Cherry (unknown variety) and Bigarra Cherry.

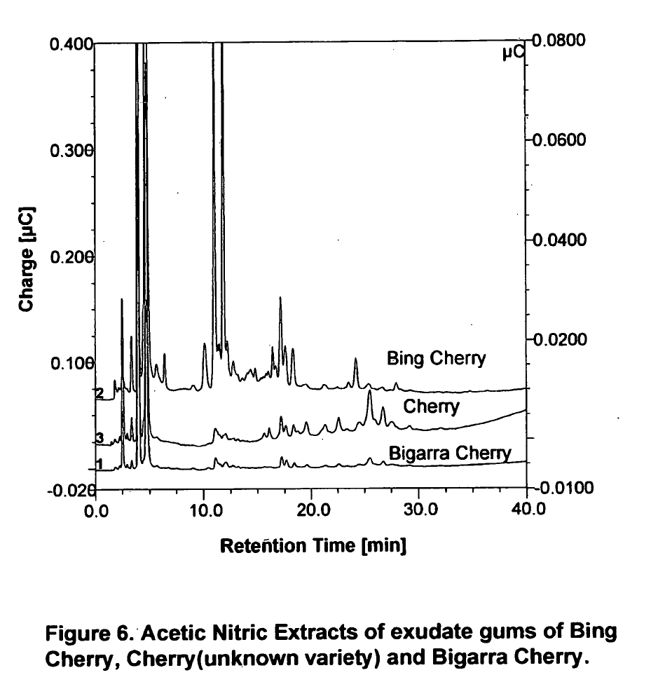

FIG. 7. Acetic Nitric extract of Lotus (Nelumbo nucifera, China Antique) seed coat.

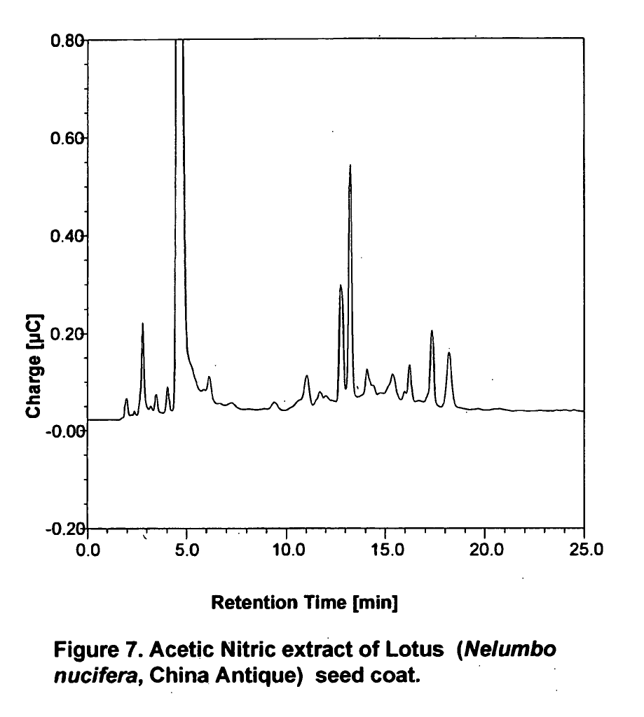

FIG. 8. Acetic Nitric extract of Lotus (Nelumbo nucifera, China Antique) cotyledons, modern and 467 year old (mirror image comparison). Differences In oligomers eluting after 10 minutes are apparent.

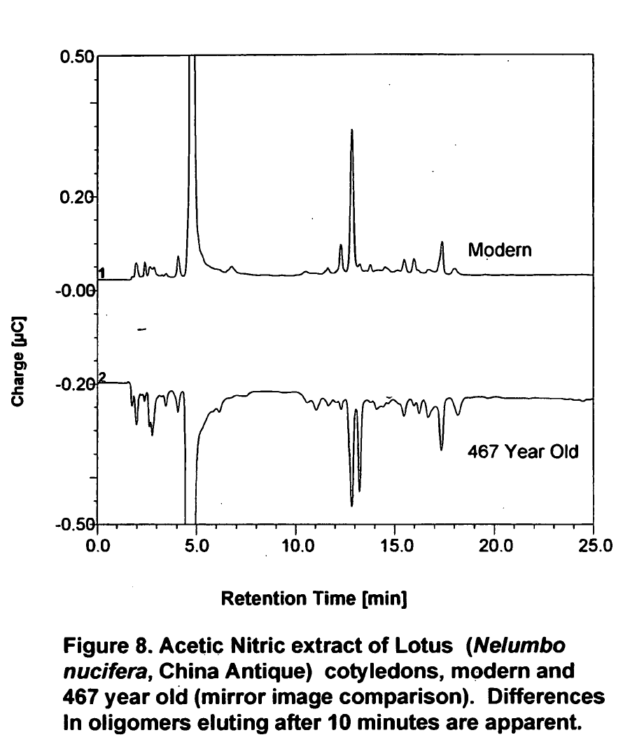

### DETAILED DESCRIPTION OF THE PREFERRED EMBODIMENTS

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide methods for determining identity and quality of plant cell wall materials, especially cotton fibers, and other cellulose containing products, such as wood and paper, through the analysis of selected polysaccharide fractions.

Cell wall biosynthesis is a highly complex process which involves soluble substrates being converted to insoluble products at the surface of the cell membrane or external to it. This is complicated by the synthesis of a primary wall followed by the synthesis of the secondary wall often with overlap of the synthesis of both. The products include polysaccharides, glycoproteins, proteins and enzymes which may exist in complexes or be covalently linked to each other. Correlations between cell growth and substrate concentrations and the activities of several enzymes have been made (Murray and Bandurski, 1975; Murray and Brown, 1997). The fact that hydrolysis of sucrose to glucose and fructose is an integral part of fiber wall synthesis (Basra et al., 1990) is consistent with findings described in the instant application. A direct relationship between cell growth and acid invertase activity has been demonstrated in several plant tissues (Morris and Arthur, 1985; Sturm and Chrispeels, 1990; Basra et. al., 1990; Sturm, et. al., 1995; Buchala, 1987). The increased invertase activity is the result of transcription of messenger RNA, rather than simply an enzyme kinetic effect, therefore, the invertase response is specific and induced (Sturm and Chrispeels, 1990; Sturm, et. al., 1995).

The secondary cell wall of cotton fibers consists almost entirely of cellulose which directs interest to cellulose biosynthesis. The potential role of sucrose synthase with the cellulose synthetic apparatus has been proposed (Amor, et. al., 1995, Delmer, 1999). The possible role of the invertase mentioned above, the possible role of lipid-bound intermediates (Matthyse, et. al., 1995; Brett, 2000) and the suggestion of self-assembly mechanisms (Brett, 2000) remain observations in search of explanations. In this specification I describe a series of glycan oligomers which appear to be associated with this cell wall biosynthetic process.

My interest is in understanding the dynamics of carbohydrate metabolism during cotton fiber development. Since a plant cell must synthesize cell wall material in order to grow and develop, knowledge of the events in cell wall biosynthesis can be used to monitor plant growth and to detect aberrations in growth due to environmental influences (Murray, 1998, 2000). The cotton fiber is unique in its development since it is a plant cell that usually does not divide or store starch. During the period of fiber elongation, it is generally synthesizing primary cell wall (Graves and Stewart, 1988). Following the period of cell elongation, the fiber cell thickens as it synthesizes secondary cell wall, which consists almost entirely of cellulose.

The glycans described below constitute another piece of the cell wall biosynthetic process. Since they can be extracted from developing cotton fibers, mature cotton fibers and aging cotton fibers in fabric, they may be subunits of the cotton fiber. Since they have been extracted from every sample of plant cell wall material examined suggests that they are fundamental elements, Which occur with cellulose.

Not only are the oligomer profiles of each source of plant material unique but the exudate gums from different species are also unique. Both the aqueous extracts and the hcl extracts are unique.

### Materials and Methods

Extraction of samples. The samples were first extracted with water at 0° to remove soluble oligosaccharides and monosaccharides (Murray, 1998). Typically, a 5 mg sample of chopped fibers was placed in a 1.7 ml screw cap plastic tube to which 0.5 ml water was added, the tube shaken, then placed in a Branson 85 W sonicator filled with ice water for 15 min Following removal of the cold water extract with a Pasteur pipette, 0.5 ml of 0.1N HCl was added and the tube was placed in a boiling water bath for 30 minutes to extract the glucose containing oliogmers (Murray, 2000). The mono- and oligosaccharides extracted by the cold water procedure include myo-inositol, galactinol, arabinose, glucose, fructose, melibiose, sucrose, manninotriose, verbascotetraose, raffinose, stachyose, verbascose and, tentatively, ajugose (Murray, 1998, 2000). The oligosaccharides extracted by the 0.1N HCl procedure can also be used as indicators of cell wall biosynthesis and fiber development (Murray, 2000). The HCl extracts were neutralized with an equivalent amount of 1N NaOH prior to HPAEC-PAD. Next the insoluble material was subjected to the extraction which is the subject of this application. 1.0 ml of acetic nitric reagent (Updegraff, 1969) [80% acetic acid, 1.8N nitric acid] was added to the tube and it was placed in a boiling water bath for 30 minutes. The acetic nitric extract was then removed with a pateur pipette and placed in another tube. The extract was then taken to dryness in a Speed-Vac to remove the acetic acid and the nitric acid. The dried material was then dissolved in 1.0 ml of water and centrifuged prior to analysis by HPAEC.

Chromatography. HPAEC-PAD was performed using a CarboPac PA-1 column. The eluent was 150 mM sodium hydroxide, isocratic from 0 to 5 min then a linear sodium acetate gradient from 5 min to 40 min going from 0 to 500 mM in 150 mM NaOH at a flow rate of 1 ml/min. The detector wave form was the following:

Waveform Time=0.00, Potential=0.10

Waveform Time=0.20, Potential=0.10, Integration=Begin

Waveform Time=0.40, Potential=0.10, Integration=End

Waveform Time=0.41, Potential=−2.00

Waveform Time=0.42, Potential=−2.00

Waveform Time=0.43, Potential=0.60

Waveform Time=0.44, Potential=−0.10

Waveform Time=0.50, Potential=−0.10

For monosaccharide composition, oliogomers were obtained by collecting fractions of the HPAEC-PAD eluent, which was passed through a Dionex Carbohydrate Membrane Desalter to remove salt. Alternatively, fractions were desalted by passing over a Dowex 50 column, ammonium form. Fractions were then lyophilized and taken up in 200 μl of water, made up to 2N trifluoroacetic acid (TFA) (Manzi and Varki, 1993). flushed with argon and sealed in screw cap plastic vials with O-rings. The samples were then placed in a heating block at 100° for 2-4 hr. Following hydrolysis, the samples were taken to dryness in a Speed-Vac overnight and then taken up in 200 μl of water for HPAEC-PAD on a Dionex CarboPac-PA10 column under isocratic conditions in 15 mM NaOH.

### Acetic Nitric Extractable Oligomers

Hydrolysis (acid) of individual peaks has demonstrated that they contain galactose, glucose and mannose. A/N instead of HCl

### Universality of the Method

The same method of extraction with hot weak acid can be applied to virtually any plant material. The pattern of oligomers released is unique for each plant and tissue and further demonstrates effects of developmental state and growth conditions. Differences in growth conditions may reflect the influence of environmental pollutants. This method of analysis can be applied to any plant material including foodstuffs. The method has been applied to food grains such as wheat, corn, rye, rice and oats. Each type of grain shows a unique profile of soluble mono- and oligosaccharides, a unique profile of oliogmers released by the hot weak acid, as well as unique profiles of the redissolved alcohol precipitates and in some cases the enzymatic digest of the redissolved alcohol precipitates.

Fiber Identification

The inventive multimer (oligomer) extraction is ideally suited for evaluating cotton fiber samples for a number of defects that plague the textile industry. Oligomer distribution data in an accessable database permits identification of cotton cultivars based on the oligomer profile of the fibers. Although presently done on a scale of identification should be feasible on a scale of 50-100 μg,

### Characterization of Oligomers

The monosaccharides contained in the acetic nitric extractable oligomers contain glucose as the major constituent. However, other components include mannose, galactose, scyllo-inositol and sorbitol although the data is not presented here

### Source Identification of Woods and Other Plant Materials

The above-described experiments indicated that plant cell wall materials such as cotton give surprisingly consistent patterns of extracted multimers. This suggested that the method might yield unique “fingerprints” that could be used for identifying the origin of cellulosic materials for forensic and other purposes (e.g., quality control of wood pulps, etc.). The present method of analysis has now been extended to a wide variety of cellulose containing materials (many of them exotic woods). Therefore, it is logical to assume that such oligomers will be released from virtually all cellulose containing materials which are derived from a plant cell wall. Each species of plant would be expected to have a slightly different array of enzymes and pool sizes of various cell wall precursors. This would lead to each type (species) of wood—essentially composed of secondary cell walls containing cellulose and lignin—having unique cellulose characteristics. In addition, analagous oligomers are found in plant tissues which are not characterized by secondary cell walls. They have been extracted from food grains such as wheat, oats, rye, barley and rice. It is quite possible and likely that these oligomers comprise that fraction of the food grains which is referred to as “soluble fiber” by the dietary field since they are likely not digested in the human gastrointestinal system.

The present invention would appear to be a more quantitative and automatic replacement for the “classical” microscopic approach of identifying wood samples. Previously a plant anatomist with considerable expertise was needed to identify small wood samples by examining microscopic cellular structures. There are a number of reasons that identification of wood samples might be required. In the case of imported wood products it might be required to demonstrate that none of the wood comes from endangered species. Some exotic wood is extremely expensive. Proof might be required that the wood is indeed of the correct, rare species. The present invention is also a quality control method for wood pulp processing. The type and quantity of multimers correlates with the degree of processing of wood pulp with the purer, higher quality pulps resulting from more extensive processing. The present method allows a given pulp sample to be rapidly and unambiguously evaluated to demonstrate pulp quality. This can be especially valuable in the formulation and quality control of material in recycled paper processing.

The oligomers utilized by the present invention appear to have a key role in the structure and synthesis of plant cell walls. The relative amounts of oligomers extracted with acetic nitric reagent increase with age of developing cotton fibers and are most abundant at maturity. The roles of UDPG (uridine diphosphate glucose), sucrose and sucrose synthase have been well described (Delmer, 1999). The influence of the concentrations of myo-inositol, sucrose, raffinose, cellobiose and glycerol on the oligomers extracted from fibers following incubation also supports the notion that a number of these sugars may function as substrates. The prospect of substrates originating external to the fiber being incorporated into the cellulose of the fiber wall was first raised by Delmer, et. al. 1974.

Clearly, biosynthesis of a polymer as large as cellulose may involve carbohydrates larger than sucrose. That such intermediates have not been described may be attributable to the complexity of carbohydrate biochemistry, and the relative fragility of glycoprotein associations, in the presence of rigorous extraction procedures. In this work, the use of mild extraction procedures, together with HPAEC-PAD, has revealed a number of, as yet not fully-characterized, oligomers. Such oligomers have been found in a number of cellulosic materials. The relative abundance of these oligomers varies with source and with developmental variables within a source. Moreover, the oligomers have been found in association with protein and, in certain experimental incubations, have behaved as if their solubility, acid-lability, and associated soluble products were affected by temperature and by amendment with biologically active saccharides. In short, they have behaved as if they were components of a biosynthetic apparatus. It is probable that the process of cellulose synthesis involves as yet un-described enzymatic activity, and that such activity is energetically favored by the conformation of glycan and glycoprotein conformations that are amenable to low-energy and possibly low-bioenergetic interconversion.

In addition to the equivalents of the claimed elements, obvious substitutions known to one with ordinary skill in the art are defined to be within the scope of the defined elements. The illustrated embodiment has been set forth only for the purposes of example and that should not be taken as limiting the invention. Therefore, it is to be understood that, within the scope of the appended claims, the invention may be practiced other than as specifically described herein.

### REFERENCES

Amor, Y., Haigler, C. H., Johnson, S., Wainscott, M., and Delmer, D. P., 1995, A Membrane-Associated Form of Sucrose Synthase and Its Potential Role in Synthesis of Cellulose And Callose In Plants. Proc. Natl. Acad. Sci. USA 92:9353-9357.
Basra, A. S., Sarlach, R. S., Nayyar, H. and Malik, C. P., 1990, Sucrose Hydrolysis in Relation to Development Of Cotton (Gossypium Spp.) Fibres, Indian Journal of Experimental Biology, 28:958-988.
Brett, C. T., 2000, Cellulose Microfibrils in Plants: Biosynthesis, Deposition, and Integration into the Cell Wall, Int. Rev. Cytology 199:161-199.
Buchala, A., 1987, Acid β-fructofuranoside Fructohydrolase (Invertase) in Developing Cotton (Gossypium arboreum L.) Fibres and its Relationship to β-glucan Synthesis from Sucrose Fed to the Fibre Apopoplast, J. Plant. Physiol. 127:219-230.
Delmer, D. P, Beasley, C. A. and Ordin, L., 1974, Utilization of Nucleoside Diphosphate Gucoses in Developing Cotton Fibers, Plant Physiol. 53:149-153.
Delmer, D. P., 1999, Cellulose Biosynthesis: Exciting Times for a Difficult Field of Study. Ann. Rev. Plant Physiol. Plant Mol. Biol. 50:245-276.
Graves, D. A., and Stewart, J. McD., 1988, Analysis of the Protein Constituency of Developing Cotton Fibres, J. Exp. Botany 39:59-69.
Manzi, A. E. And Varki, A, 1993, Compositional Analysis Of Glycoproteins, In Glycobiology: A Practical Approach, Eds. M. Fukuda And A Kobata, Pp. 27-77, Oxford University Press. Matthysse, A. G., Thomas, D. L., and White, A. R., 1995, Mechanism of Cellulose Synthesis in Agrobacterium tumefaciens, J. Bact. 117:1076-1081.
Meinert, M. C. and Delmer, D. P., 1977, Changes in Biochemical Composition of the Cell Wall of the Cotton Fiber During Development, Plant Physiol. 59, 1088-1097.

Morris, D. A. and Arthur, E. D., 1985, Invertase Activity, Carbohydrate Metabolism and Cell Expansion in the Stem of Phaseolus vulgaris L. J. Exptl. Bot. 36:623-633.
Murray, A. K. and Brown, J., 1997, Glycoconjugate Profiles of Developing fibers from Different Fruiting Branches on the Same Plant, 1997 Proceedings Beltwide Cotton Conferences, p. 1496-1499.
Murray, A. K., 1998, Method For Monitoring Growth And Detection Of Environmental Stress In Plants, U.S. Pat. No. 5,710,047.
Murray, A. K., 2000, Method For Detecting Growth And Stress In Plants, U.S. Pat. No. 6,051,435.
Murray, Allen K, 2003, Method for monitoring textile fiber quality, and for analysis and identification of paper, wood and other cellulose containing materials, U.S. Pat. No. 6,562,626, issued May 13, 2003.
Murray, Allen K and Robert L. Nichols, 2004, Strategies for Biochemical Characterization of Cotton Fibers, Proceedings of the World Cotton Research Conference 3, Cape Town, South Africa 2003, 1457-1465, Agricultural Research Council—Institute for Industrial Crops, Pretoria, South Africa. Publisher: Agricultural Research Council—Institute for Industrial Crops
Murray, Allen K, 2004, Method For Monitoring Textile Fiber Quality, Analysis And Identification Of Paper, Wood, Grains, Foods And Other Cellulose Containing Materials Using Glycan Oligomer Analysis, U.S. Patent Application Publication, US 2004/0152201 A1, Aug. 5, 2004.
Murray, A. K. and Bandurski, R. S., 1975. Correlative Studies on Cell Wall Enzymes and Growth. Plant Physiology 56:143-147.
Murray, Allen K, Robert L. Nichols, and Gretchen F. Sassenrath-Cole, 2001, Cell Wall Biosynthesis: Glycan Containing Oligomers in Developing Cotton Fibers, Cotton Fabric, Wood and Paper, Phytochemistry, In Press.
Sturm, A. and Chrispeels, M. J., 1990, cDNA Cloning of Carrot Exocellular β-Fructofuranosidase and Its Expression in Response to Wounding and Bacterial Infection, The Plant Cell 2:1107-1119.

Sturm, A., \check{S}ebková, V., K., Lorenz, Hardegger, M., Lienhard, S., and Unger, C., 1995, Development- and organ-specific expression of the genes for sucrose synthase and three isoenzymes of acid β-frucdtofuranosidase in carrot, Planta 195:601-610.

Updegraff, D. M., 1969, Semi-micro determination of cellulose in biological materials. Anal. Biochem. 32:420-424.

I claim:

1. A method of analyzing samples of textiles, wood pulp and plant products comprising the steps of:
producing a cold water extract by extracting the samples with cold water,
treating insoluble materials from the cold water extract step with dilute hot acid to yield an acid extract;
neutralizing the acid extract;
treating the neutralized acid extract with acetic nitric reagent (80% acetic acid, 1.8N nitric acid) at 100° C. for 30 minutes, taken to dryness and made up into an aqueous solution; and
analyzing the aqueous solution to reveal a carbohydrate multimer pattern.

2. The method of analyzing of claim 1, further comprising the step of analyzing soluble mono- and oligosaccharides contained in the cold water extract and the oligomers in the neutralized dilute acid extract;

3. A method to identify the species of a sample of wood or other cellulosic material of plant origin comprising the steps of:
extracting specimens of known species of wood or cellulosic material with dilute acetic nitric acid to produce known extracts;
analyzing each known extract to reveal a pattern of carbohydrate multimers diagnostic of the species or cultivar from which the extract was made;
extracting the sample of wood or cellulosic material with dilute hot acid to produce a sample extract;
analyzing the sample extract to reveal a pattern of carbohydrate multimers characteristic of the sample extract; and
comparing the pattern of the sample extract to the patterns of the known extract to determine the species or cultivar of the sample.

4. A generally applicable method for characterization and identification of carbohydrate containing materials which may or may not contain cellulose such as exudate gums from plants as well as non-woody plant material such as cotyledons as constituents of foods or other items such as works of art.
